(19) United States
(12) Patent Application Publication
(10) Pub. No.: US 2011/0098246 A1
(43) Pub. Date: Apr. 28, 2011

(54) METHOD FOR PREVENTING CORTICOSTEROID USAGE

(75) Inventors: Bernd Stahl, Rosbach (DE); Jürgen Jelinek, Rosbach (DE); Günther Boehm, Echzell (DE)

(73) Assignee: N.V. Nutricia

(21) Appl. No.: 12/996,344

(22) PCT Filed: Jun. 5, 2009
(86) PCT No.: PCT/NL2009/050311

§ 371 Date: Dec. 3, 2010

(30) Foreign Application Priority Data
Jun. 6, 2008 (EP) 08157767.8

(51) Int. Cl. A61K 31/702; A61P 17/00; A61P 17/06

(52) U.S. Cl. 514/54

(57) ABSTRACT

The present invention relates to the use of nutritional compositions comprising non-digestible oligosaccharides for preventing corticoid administration, particularly to infants.

### FIELD OF THE INVENTION

The present invention relates to the use of nutritional composition for preventing corticoid administration, particularly to infants.

### BACKGROUND OF THE INVENTION

Skin diseases such as eczema, infantile eczema, atopic dermatitis, dermatitis herpetiformis, contact dermatitis, seborrhoeic dermatitis, neurodermatitis, psoriasis and intertrigo are a significant problem. For the treatment of such skin diseases corticosteroids are used in humans. However, the use of coticosteroids has important drawbacks, as these may cause steroid-induced side effects. Some steroid-induced side effects are cutaneous changes such as skin blanching from acute vasoconstriction, hypo-pigmentation, rebound worsening of the pre-existing skin condition, miliaria, rosacea, perioral dermatitis, acne, skin atrophy with telangiectasia, stellate pseudoscars, purpura, striae, delayed wound healing, hyper-trichosis of face; or cutaneous infections.

Bukutu et al (Pediatr. Rev. 2007; 28 (12); e87-e94) reported that a Chinese herbal medical concoction demonstrated reduced topical corticosteroid usage. Passeron et al (Allergy 2006: 61: 431-437) described a study wherein Lactobacillus rhamnosus Lcr35 plus prebiotic preparation or a prebiotic preparation derived from the fermentation broth for L. rhamnosus Lcr35 was given to children aged two and over and reported that the use of topical treatments had significantly decreased at the end of the study.

WO 2004069156 relates to formulations comprising inactivated probiotic bacteria, and treatment methods using these formulations.

WO 2005039597 relates to a method for enhancing the immune system and the treatment and/or prevention of immune system related disorders in a mammal, particularly newborns, comprising the administration of acid oligosaccharide and neutral oligosaccharide. Food compositions suitable for use in such methods are also provided.

### SUMMARY OF THE INVENTION

In a double blind, placebo controlled study in 1000 infants it was found that the administration of certain prebiotics reduced the use of corticosteroid and other dermatological preparations. In the control group, the use of corticosteroids and dermatological preparations was observed in 40 infants, whereas only 16 infants of the prebiotic group used corticosteroids and dermatological preparations. Hence, the present inventors have found that the administration of galactooligosaccharides, fructooligosaccharides, uronic acid oligosaccharides or a combination thereof effectively reduces the use of corticosteroids.

### DETAILED DESCRIPTION OF PREFERRED EMBODIMENTS

The present invention concerns a method for preventing topical corticosteroid administration and/or preventing calcineurin inhibitor administration, said method comprising administering a composition comprising at least one non-digestible saccharide selected from the group consisting of galactooligosaccharide, fructooligosaccharide, uronic acid oligosaccharide.

Also the invention concerns the use of a composition comprising at least one non-digestible saccharide selected from the group consisting of galactooligosaccharide, fructooligosaccharide, uronic acid oligosaccharide in the preparation of a composition for preventing topical corticosteroid administration and/or preventing calcineurin inhibitor administration. In other words the invention concerns at least one non-digestible saccharide selected from the group consisting of galactooligosaccharide, fructooligosaccharide, uronic acid oligosaccharide for preventing topical corticosteroid administration and/or preventing calcineurin inhibitor administration. Also the invention concerns a method for the preparation of a composition for preventing topical corticosteroid administration and/or preventing calcineurin inhibitor administration, said method comprising including at least one non-digestible saccharide selected from the group consisting of galactooligosaccharide, fructooligosaccharide, uronic acid oligosaccharide in said composition.

In the context of this invention, preventing topical corticosteroid administration preferably means that compared to a control group less individuals require the administration topically of corticosteroids for the treatment of skin diseases such as for example eczema, infantile eczema, atopic dermatitis, dermatitis herpetiformis, contact dermatitis, seborrhoeic dermatitis, neurodermatitis, psoriasis and intertrigo. In one aspect the present invention thus reduces the risk, preferably in infants, of the occurrence of side effects of the use of corticosteroids such as for example skin blanching from acute vasoconstriction, hypo-pigmentation, rebound worsening of the pre-existing skin condition, miliaria, rosacea, perioral dermatitis, acne, skin atrophy with telangiectasia, stellate pseudoscars, purpura, striae, delayed wound healing, hyper-trichosis of face; or cutaneous infections. Also, in the context of this invention, preventing calcineurin inhibitor administration typically means that compared to a control group less individuals require the administration of calcineurin inhibitor, as an alternative to the use of corticosteroids, for the treatment of skin diseases. The present invention thus typically reduces the risk, preferably in infants, of the occurrence of side effects of the use of calcineurin inhibitors. As such the present invention may be considered non-therapeutic.

### Non-Digestible Oligosaccharides

The present composition preferably comprises at least one non-digestible oligosaccharide selected from the group consisting of galacto-oligosaccharides, fructo-oligosaccharides and uronic acid oligosaccharides.

The term “oligosaccharide” as used in the present invention preferably refers to a saccharide with a degree of polymerization (DP) of 2 to 250, preferably a DP 2 to 100, more preferably 2 to 60. It is understood that in the context of this invention a saccharide with a DP in a certain range may include a mixture of saccharides with different average DP's, for example, if an oligosaccharide with a DP of 2 to 100 is included in the present composition, this may include compositions which contain oligosaccharides with a DP between 2 and 5, a DP between 50 and 70 and a DP of 7 to 60.

The term “non-digestible oligosaccharide” as used in the present invention refers to oligosaccharides which are not or only partially digested in the intestine by the action of acids or digestive enzymes present in the human upper digestive tract (small intestine and stomach) but which are fermented by the human intestinal flora. For example, sucrose, lactose, maltose and maltodextrins are considered digestible. For example, galactooligosaccharides, fructooligosaccharides and uronic acid oligosaccharides are considered non-digestible oligosaccharide.

The present composition preferably contains fructooligosaccharide. The term “fructo-oligosaccharide” as used herein refers to a non-digestible polysaccharide comprising a chain of at least 2 β-linked fructose units, with a DP of 2 to 250, preferably 7 to 100, more preferably 20 to 60. Preferably inulin is used. Inulin is for example available under the tradename “Raftilin HP®”, (Orafti). The average DP of the present fructo-oligosaccharide is preferably at least 7, more preferably at least 10, preferably below 100. The fructo-oligosaccharide used preferably has the (majority of) fructose units linked with a β(2→1) linkage. Other terms for fructooligosaccharides include inulin, fructopolysaccharide, polyfructose, fructans and oligofructose. The present composition preferably comprises fructo-oligosaccharides with a DP of 2 to 200.

The present composition preferably contains galacto-oligosaccharide. The term “galacto-oligosaccharide” as used herein refers to a non-digestible oligosaccharide, wherein at least 30% of the saccharide units are galactose units, preferably at least 50%, more preferably at least 60%. The present composition preferably comprises galacto-oligosaccharides with a DP of 2 to 100, more preferably a DP of 2 to 10. Preferably the saccharides of the galacto-oligosaccharide are β-linked. Hence, preferably the present composition comprises beta-galactooligosaccharides. In a particularly preferred embodiment the present composition comprises transgalacto-oligosaccharide ([galactose]ₙ-glucose; wherein n is an integer between 1 and 60, i.e. 2, 3, 4, 5, 6, . . . , 59, 60; preferably n is 2, 3, 4, 5, 6, 7, 8, 9 and/or 10). Transgalacto-oligosaccharides (TOS) are for example sold under the trademark Vivinal™ (Borculo Domo Ingredients, Netherlands).

The present composition preferably comprises galacto-oligosaccharides and fructo-oligosaccharides, more preferably transgalacto-oligosacharides with a DP of 2 to 7 and fructo-oligosaccharides with a DP of 10 to 100.

The present composition preferably comprises uronic acid oligosaccharide. The term uronic acid oligosaccharide as used in the present invention refers to an oligosaccharide wherein preferably at least 25%, preferably at least 50% of the monosaccharide units present in the oligosaccharide is one selected from the group consisting of guluronic acid, mannuronic acid, iduronic acid, riburonic acid, galacturonic acid and glucuronic acid. In a preferred embodiment the uronic acid oligosaccharide comprises at least 50% galacturonic acid based on total uronic acid units in the uronic acid oligosaccharide. The uronic acid oligosaccharides used in the invention are preferably prepared from pectin, pectate, alginate, chondroitine, hyaluronic acids, heparine, heparane, bacterial carbohydrates, sialoglycans, fucoidan, fuco-oligosaccharides and/or carrageenan, more preferably from pectin and/or alginate, even more preferably from pectin, most preferably polygalacturonic acid. The present uronic acid oligosaccharide is preferably a pectin degradation product and/or alginate degradation product. Preferably the pectin degradation product is a pectin hydrolysate (prepared by hydrolysis) and/or pectin lyasate (prepared by beta-elimination). The pectin degradation product is preferably prepared from fruit and/or vegetable pectin, more preferably apple pectin, citrus pectin and/or sugar beet pectin, more preferably from apple, citrus and/or sugar beet pectin. The pectin degradation product is preferably prepared with lyases and/or variations of the temperature and pressure, more preferably by beta-elimination. The pectin degradation product is preferably a pectin lyasate.

Preferably the present composition comprises uronic acid oligosaccharide with a DP of 2 to 250, more preferably a DP of 2 to 100, even more preferably a DP of 2 to 50, most preferably a DP of 2 to 20. Preferably the present composition comprises between 25 and 100 wt. %, more preferably between 50 and 100 wt. % uronic acid oligosaccharide with a DP of 2 to 250 based on total weight of uronic acid in the composition, more preferably a DP of 2 to 100, even more preferably a DP of 2 to 50, most preferably a DP of 2 to 20.

The present uronic acid oligosaccharide is preferably obtainable by enzymatic digestion of pectin with pectin lyases, pectate lyase, endopolygalacturonase and/or pectinase.

In a preferred embodiment at least one of the terminal hexuronic acid units of the uronic acid oligosaccharide has a double bond, which is preferably situated between the C₄ and C₅ position of the terminal hexuronic acid unit. The double bond effectively protects against attachment of the pathogenic bacteria to the intestinal epithelial cells. Preferably one of the terminal hexuronic acid units comprises the double bond. The double bond at terminal hexuronic acid unit can for example be obtained by enzymatic hydrolysis of pectin with lyase.

The present composition preferably comprises between 0.01 and 10 g uronic acid oligosaccharide with a DP of 2 to 250, preferably a DP of 2 to 100, per 100 g dry weight of the present composition, more preferably between 0.05 and 6 g, even more preferably 0.2 to 2 g per 100 g dry weight. The present composition preferably comprises between 0.01 and 10 g galacturonic acid oligosaccharide with a DP of 2 to 250, preferably a DP of 2 to 100, per 100 g dry weight of the present composition, more preferably between 0.05 and 6 g, even more preferably 0.2 to 2 g.

The present composition thus preferably comprises different non-digestible oligosaccharides with different degrees of polymerization (DP). In terms of fractions of oligosaccharides with different ranges of DPs, the composition preferably has the following weight ratios:
a. (non-digestible oligosaccharides with DP 2 to 5): (non-digestible oligosaccharides with DP 6, 7, 8, and/or 9)>1; and/or
b. (non-digestible oligosaccharides with DP 10 to 60): (non-digestible oligosaccharides with DP 6, 7, 8, and/or 9)>1
   Preferably both weight ratios are above 2, even more preferably above 5.

The present composition preferably comprises 0.5 to 75 g of the non-digestible oligosaccharides per 100 g dry weight, preferably between 0.5 and 50 g. The present composition preferably comprises 0.1 to 75 g of the galacto-oligosaccharides per 100 g dry weight, preferably between 0.1 and 50 g.

The present method preferably comprises the administration of a serving comprising between 0.05 and 25 g non-digestible oligosaccharide, preferably between 0.1 and 5 g.

The present method preferably comprises the administration of a serving comprising between 0.05 and 25 g galacto-oligosaccharides, preferably between 0.1 and 5 g galacto-oligosaccharides.

### Corticosteroid & Calcineurin Inhibitor

The present invention relates to preventing topical corticosteroid administration and/or preventing calcineurin inhibitor administration.

Preferably the administration of one or more of following corticosteroids is prevented: alclometasone dipropionate, amcinonide, beclamethasone dipropionate, betamethiasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone preparations, fluprednidene acetate, flurandrenolone, Fluticasone propionate, halcinonide, halobetasol propionate, hydrocortisone, hydrocortisone acetate, hydrocortisone butyrate, hydrocortisone valerate, methylprednisolone acetate, mometasone furoate and triamcinolone acetonide and mixtures thereof.

Calcineurin inhibitors are immunosuppressant agents originally developed for systemic administration to prevent allogeneic transplant rejection. These agents inhibit calcineurin in the skin, which blocks early T-cell activation and the release of cytokines. Topical formulations were developed as alternatives to topical corticosteroids. Preferably the use of one or more of following calcineurin inhibitors is prevented: pimecrolimus, tacrolimus and mixtures thereof.

### Infant

Infants frequently suffer from skin diseases. Administration of calcineurin inhibitors to infants is not recommended. Topical administration of corticosteroids to infant, particularly young infants, also has undesirable side effects. Hence, it is particularly desired to reduce the administration of such medicaments to infants.

The present inventors found that the ingestion of the present composition prevents the topical administration of corticosteroid and other dermatological preparations in infants with an age up to one year. Hence the present invention is particularly advantageous for infants with an age between 0 and 24 months, more preferably with an age between 0 and 12 months, or in one embodiment with an age between 0 and 6 months.

### Compositions

The present composition is preferably enterally administered, more preferably orally.

The present composition is preferably a nutritional formula, preferably an infant formula. The present composition can advantageously be applied as a complete nutrition for infants. The present composition preferably comprises lipid, protein, and carbohydrate and is preferably administered in liquid form. The present invention includes dry compositions, e.g. powders, which are accompanied with instructions as to admix said dry compositions, in particular nutritional formula, with a suitable liquid, e.g. water.

The present invention advantageously concerns a composition wherein the lipid provides 5 to 50% of the total calories, the protein provides 5 to 50% of the total calories, and the carbohydrate provides 15 to 90% of the total calories. Preferably, in the present composition the lipid provides 35 to 50% of the total calories, the protein provides 7.5 to 12.5% of the total calories, and the carbohydrate provides 40 to 55% of the total calories. For calculation of the % of total calories for the protein component, the total of energy provided by the proteins, peptides and amino acids needs to be taken into account.

The present composition preferably comprises at least one lipid selected from the group consisting of animal lipid (excluding human lipids) and vegetable lipids. Preferably the present composition comprises a combination of vegetable lipids and at least one oil selected from the group consisting of fish oil, animal oil, algae oil, fungal oil, and bacterial oil. The present composition comprising non-digestible oligosaccharides excludes human milk. The protein component used in the nutritional preparation are preferably selected from the group consisting of non-human animal proteins (preferably milk proteins, preferably proteins from cow's milk), vegetable proteins (preferably soy protein and/or rice protein), free amino acids and mixtures thereof. The present composition preferably contains casein, whey, hydrolysed casein and/or hydrolysed whey protein. Preferably the protein comprises intact proteins, more preferably intact bovine whey proteins and/or intact bovine casein proteins. As the present composition is preferably suitably for use by infants suffering from allergy, the protein is preferably selected from the group consisting of hydrolyzed milk protein.

The liquid nutritional composition preferably has a caloric density between 0.1 and 2.5 kcal/ml, even more preferably a caloric density of between 0.5 and 1.5 kcal/ml, most preferably between 0.6 and 0.8 kcal/ml.

Particularly the present invention provides a composition as described herein above accompanied with indications (e.g. written material) comprising statement that the administration of the composition (e.g. to the infant) prevents the incidence of corticosteroid usage; reduces the duration of corticosteroid use and/or reduces the chance that the subject will need topical corticosteroid to prevent skin diseases.

### EXAMPLES

### Example 1

### Clinical Study

In a randomized controlled double blind European multi-centre trial (7 centres in 5 countries) 1187 healthy term infants without family history of atopy were recruited receiving either a formula supplemented with the present prebiotic mixture (galactooligosaccharides, fructooligosaccharides and pectin derived oligosaccharides, 8 g/l formula), a standard formula (control) or breast milk (the latter not randomized). A number of 186 infants (15.7%) dropped out (no group difference). A number of 835 infants ran through the study per protocol, i.e. were completers and followed the feeding scheme correctly: 292 in the new prebiotic group, 300 in the control group, and 243 in the reference group. The use of corticosteroids and dermatological preparations was observed in the FAS analysis in 16 infants of the prebiotics group, whereas this was observed in 40 infants of the control group (Fisher p=0.0018) (PPS 10 versus 29 p=0.0025).

### Example 2

### Composition

An infant formula composition comprising per 100 ml ready to feed formula: 1.6 g protein, 3.6 g fat, 6.4 g digestible carbohydrates (mainly lactose), 0.8 g non-digestible oligosaccharides of which 0.54 g transgalacto-oligosaccharides, 0.06 g inulin, and 0.2 g pectin hydrolysate (prepared by lyase treatment of citrus pectin).

1.-6. (canceled)

7. A method of reducing or preventing the incidence of skin disease in a mammal, comprising administering to a mammal in need thereof a composition comprising galactooligosaccharide, fructooligosaccharide, and uronic acid oligosaccharide.

8. The method of claim 7, wherein the mammal is an infant between 0 and 12 months of age.

9. The method according to claim 7, wherein the composition is orally administered.

10. The method according to claim 7, wherein the skin disease is eczema, infantile eczema, atopic dermatitis, dermatitis herpetiformis, contact dermatitis, seborrhoeic dermatitis, neurodermatitis, psoriasis and intertrigo.

11. The method of claim 7, wherein the composition further comprises, based on the total calories of the composition:
(a) 5 to 50% calories from lipid;
(b) 5 to 50% calories from protein;
(c) 15 to 90% calories from carbohydrate.

12. The method according to claim 7, wherein the uronic acid oligosaccharide is prepared from pectin.

13. The method according to claim 7, wherein the corticosteroid is selected from the group consisting of alclometasone dipropionate, amcinonide, beclamethasone dipropionate, betamethiasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone preparations, fluprednidene acetate, flurandrenolone, fluticasone propionate, halcinonide, halobetasol propionate, hydrocortisone, hydrocortisone acetate, hydrocortisone butyrate, hydrocortisone valerate, methylprednisolone acetate, mometasone furoate, triamcinolone acetonide, pimecrolimus, tacrolimus and mixtures thereof.

14. A method of reducing the duration of administration of a topical corticoid steroid or a calcineurin inhibitor to treat a skin disease in a mammal, comprising administering to a mammal being treated with a topical corticoid steroid or a calcineurin inhibitor a composition comprising galactooligosaccharide, fructooligosaccharide, and uronic acid oligosaccharide.

15. The method of claim 14, wherein the mammal is an infant between 0 and 12 months of age.

16. The method according to claim 14, wherein the composition is orally administered.

17. A method of reducing the occurrence of side effects from the use of corticosteroids or calcineurin inhibitors, comprising administering to a mammal being treated with a topical corticosteroid or calcineurin inhibitors a composition comprising galactooligosaccharide, fructooligosaccharide, and uronic acid oligosaccharide.

18. The method of claim 17, wherein the mammal is an infant between 0 and 12 months of age.

19. The method according to claim 17, wherein the composition is orally administered.

20. The method according to claim 17, wherein the side effect is selected from the group consisting of skin blanching from acute vasoconstriction, hypo-pigmentation, rebound worsening of the pre-existing skin condition, miliaria, rosacea, perioral dermatitis, acne, skin atrophy with telangiectasia, stellate pseudoscars, purpura, striae, delayed wound healing, hyper-trichosis of face; or cutaneous infections.
